(19) United States
(12) Patent Application Publication
(10) Pub. No.: US 2004/0140347 A1
(43) Pub. Date: Jul. 22, 2004

(54) Sanitizing secure and safe mail box

(76) Inventors: Gueorgui Mihaylov, Virginia Beach, VA (US); Evdokia Mihaylov, Virginia Beach, VA (US)

Correspondence Address: GUEORGUI MIHAYLOV, 1745 LAKE CHRISTOPHER DRIVE, VIRGINIA BEACH, VA, 23464, US

(21) Appl. No.: 10/672,042

(22) Filed: Sep. 25, 2003

Related U.S. Application Data
(63) Continuation-in-part of application No. 10/094,839, filed on Mar. 11, 2002, now Pat. No. 6,655,577.

(51) Int. Cl. B65D 91/00

(52) U.S. Cl. 232/31

(57) ABSTRACT

A mailbox is developed which comprises a separated top and bottom port. A pivotally hinged gate/tray serves for depositing the incoming mail. The gate/tray has a pocket for outgoing mail and secures the top port from theft. Under the gate/tray a fixture with short wave ultraviolet (UV) light irradiates the box inside simultaneously generating ozone for a predetermined time. This time starts after the outgoing mail is put in the pocket or the incoming mail is dropped inside. A specially designed basket provides space around the mail where sanitizing gas freely diffuses. The light source can irradiate and activate gas-releasing objects generating other disinfecting gases such as chlorine, chlorine dioxide and ethylene oxide. The bottom port is closed by a locked door and allows retrieving the mail, and setting and maintaining the UV fixture. Two signals on an outside flag indicate the beginning and the end of the sanitizing process.

### CROSS-REFERANCE TO RELATED APPLICATION

This application is Continuation-in-part (CIP) of Ser. No. 10/094,839 filed Mar. 11, 2002 by the present inventors.

### BACKGROUND OF THE INVENTION

1. Field of the Invention

The present invention relates to the field of mail boxes, more specifically to the secured and safe mail boxes and further particularly to the sanitizing mailboxes.

2. Description of the Prior Art

The notion of secured mailbox is the driving force for the variety of mailboxes. Taking apart the constructions in which the security-locking device is used, because of their obviousness, one common approach is to use constructions applying two separate housings divided by secured moving part or space as in the following U.S. Pat. Nos. 827,482; 830,231; 1,202,251; 1,432,843; 2,602,584; 3,735,919; 5,000,378; 5,148,947; 5,938,113; 6,247,642.

The use of the means resembling revolving door as security closing door is in the base of U.S. Pat. Nos. 830,231; 5,482,208; 5,938,113; 6,247,642. This usage leads to constructions providing needed security, but not always adequate. Even in the most recent U.S. Pat. No. 6,247,642 of Wilson L. the solution for outgoing mail is mentioned only as wish, rather than designed as a construction.

Although the safety of the mail from the influence of the elements should be always the center of attention, but it has not been particular matter in the prior art. Most of the inventions consider those problems solved by the virtue of the concept of closed box. Cascading water, melting snow or condensation of water vapors inside of the box can readily have some negative consequences according to different designs of above mentioned inventions as well as in most of the mail boxes recently marketed.

Whereas a lot of diseases could be spread by bacteria-contaminated surfaces including mail of any kind, none of the prior art inventions relates to sanitizing mailbox for the incoming and outgoing mail by use of any means. On the other hand there are known devices for sanitizing the airflow such as U.S. Pat. Nos. 4,227,446; 5,523,057; 5,558,158; 5,635,133; 5,656,242 although none of them is providing sanitizing of a surface of object, more specifically surface of mail.

The U.S. Pat. No. 5,526,979 issued to Mann is claiming outgoing mail pocket dimensionally equal to approximately one half of the upper door used as incoming mail tray, therefore allowing only ½ of the size of outgoing compare to incoming mail. The pocket is limited by the upper door and is inconvenient for small envelopes. The incoming mail goes directly to the very bottom of the box where it could be exposed to the condensed or otherwise penetrated water. The incoming mail can easily slide down in outgoing pocket, which defeats the purpose of this design.

In the U.S. Pat. No. 6,244,505B1 to Grimes et al. describes a mail tray that when it is open can stop the ingoing mail from falling down. There is no means to drain the condensed water.

Others U.S. Pat. Nos. 3,874,583 to Moll, 5,979,751 to Maddox, 6,299,061B1 to Henson, although they secure the mail, they have no means for outgoing mail and water protection.

Banerjea in his U.S. Pat. No. 6,119,622 is teaching pickup light indicator, which is turned “on” when the mail is retrieved from the box.

In the U.S. Pat. Nos. 1,432,843 to Dooley and 5,335,317 to Sokolowski the previous art is implementing flags to aware the user for incoming/outgoing mail.

Wang et al. in Pub.No US2003/0103866A1 suggest the use of UV light and special mail holder rotated by a rotor. The ozone-generation is not implied but it is still possible. The construction is bulky and inconvenient, there is no indication how the mail is secured over the shelves of the holder and how the UV light reaches it. According to the drawings the UV beams are parallel to the surface of the mail e.g. the mail is self-shading.

The use of ozone generated by strong UV sources for sanitizing mail box is indicated also in publication “The self-decontaminating mailbox” on http://web.archive.org/web/20011226171545/http://astrotoo.com/index.html.

In Pub.No US2003/0124025A1 Mize et al. suggest a biological safe mailbox claims use of gas released into the enclosed structure in the form of fog. To cover the mail a surface is sprayed wet with bleach and after that is dried. There is no suggested means to spray all surfaces of the mail especially when it is staggered and to dry them after. Any “fog” under which we have to understand aerosol has diffusion coefficient several orders of magnitude smaller than the diffusion coefficient of a gas and cannot penetrate through the envelope to sanitize the inside mail surfaces. Therefore only some cross-contamination could be avoided.

In the testimony of C. N. Haas, L. D. Betz Professor of Environmental Engineering at Drexel University before The Committee of Science, US House of Representatives on Nov. 8, 2001, the chlorine dioxide gas has been recommended as a top biological decontaminating agent. In the medical industry the use of ethylene dioxide for the same purpose is well known. The germicidal properties of hypochlorites and peroxides are broadly used for sanitizing purposes.

A main objective of the present invention is to provide a mail box having a sanitizing capabilities, whereby eliminating or drastically decreasing the effect of possible presence of bacteria, viruses or spores on the surface of the incoming and outgoing mail.

Another objective of the present invention is to ensure security of the incoming mail until it is retrieved from the owner.

Objective of the present invention is also to prevent the mail from any influence of the elements and their side effects as water condensation on inside surfaces.

Yet another objective of the present invention is to furnish the necessary level of safety to the user accomplishing aforementioned objectives.

### SUMMARY OF THE INVENTION

According to the set heretofore objectives the present inventions includes a box with two main ports—one mounted in the top part of the box closed with horizontally pivoted self closing gate/tray for incoming/outgoing mail and another one in the bottom part closed with secured door for mail retrieving. The top part of the box is furnished with means for generating short wave ultraviolet light (UV light) and respectively ozone whereby both of those agents act as sanitizers over the surface and [even] deeper into the material of the mail. In close proximity to the UV source are placed one or more holder-beds, which are filled with chemically active materials. These beds are placed the way that provide them with direct exposure to UV light and exposure to the infrared (IR) radiation and convectional heat generated by the UV source. The number of the beds can vary from one to five. They can contain in solid or adsorbed on carrier surfaces one ore more of the following chemical ingredients: alkaline chlorates, alkaline perchlorates, alkaline hypochlorites, chloramines as well as permeations tubes with high pressurized gases such as ethylene oxide or chlorine. The bottom part of the box is provided with grid-type basket allowing direct exposure of the mail to the UV light and by convection and diffusion free exposure to the ozone generated by the UV source. The basket is designed to assure a distance between mail dropped and all inside of the box walls. The bottom wall of the mailbox is equipped with drain/ventilation aperture normally covered by spring-operated cover. As set heretofore the box secure the incoming mail by self-closing gate pivoted horizontally and providing limited angle of rotation. The gate is hinged to the box body along the main axis of rotation. The main axis of gate rotation is coaxial to the cylindrical shaped hood covering the gate and forming a small gap around the rotated gate. This construction do not allows the mail once dropped, to be retrieved from the same port. The inside portion of the gate has a pocket for the outgoing mail shaped as hard-core book cover and providing “closed” position when is empty and gradually opening upon the quantity of the outgoing mail. When the pocket is in “closed” position and horizontal, the top surface of the gate/tray is flat. This position of the gate/tray is considered “open box” position and the gate/tray is ready to be used as ingoing mail support. When moving the gate to vertical position, the mail slides from the surface of the gate/tray into grid or net type structure of the basket. The basket is design the way to provide a distinct distance from the bottom surface of the box and to assure air gap wrapping the entire pail of mail.

Further in accordance with the set objectives, inside the box there is a source of UV light with wavelength approximately 254 nanometers. The light source could be rechargeable battery supplied, or permanently wired to AC source through safety electric supplier. The light is directed onto the surface of the dropped mail, which provides direct extinction of the surface bacteria, viruses and spores. The UV light also produces ozone known as very strong disinfector over a certain concentration and which concentration can be easily achieved in the confined space of the box. The generated ozone diffuses into the air surrounding the mail destroying mentioned biological hazard. The coefficient of diffusion of the ozone is high enough to allow penetration even through paper material and to act as a disinfector even inside the enveloped mail. The heat generated from the UV lamp prevents water from condensation when the incoming mail is colder than air in the boxed space until the temperature of the air and the mail equilibrates. The working time of UV lamp can be regulated by different means such as preset time relay, preset electromechanical clock mechanism or similar. Those means are activated by contact switch when the mail is dropped into the box and the gate/tray get fixed in “closed box” position. The space between the bottom of the box and the mail over the grid or net prevents the mail from eventual contact with any water collected on the bottom by previous condensation or blown by the wind as snow or rain. The bottom surface of the box is slightly sloped to one end where eventually collected water could be drained by drain/ventilation aperture.

### BRIEF DESCRIPTION OF THE DROWINGS

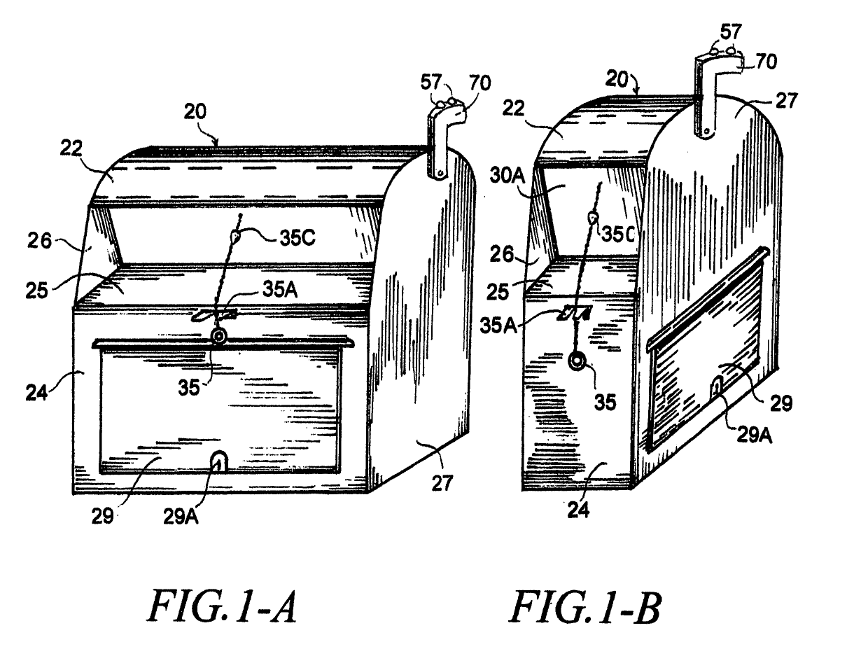

FIGS. 1-A,B are perspective view of the secured safe sanitizing mail box of the present invention having retrieving door at the front FIG. 1-A or at one of the side walls FIG. 1-B;

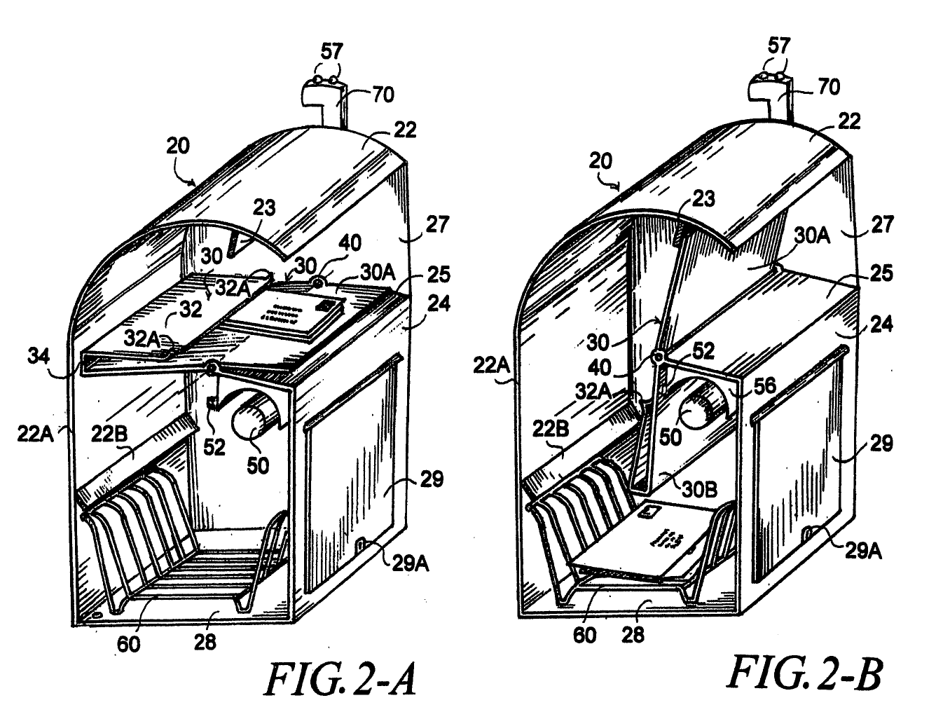

FIGS. 2-A,B are cross-sectional perspective view of the box thereof showing the two main positions of the pocket/gate charged with outgoing FIG. 2-A and incoming FIG. 2-B mail;

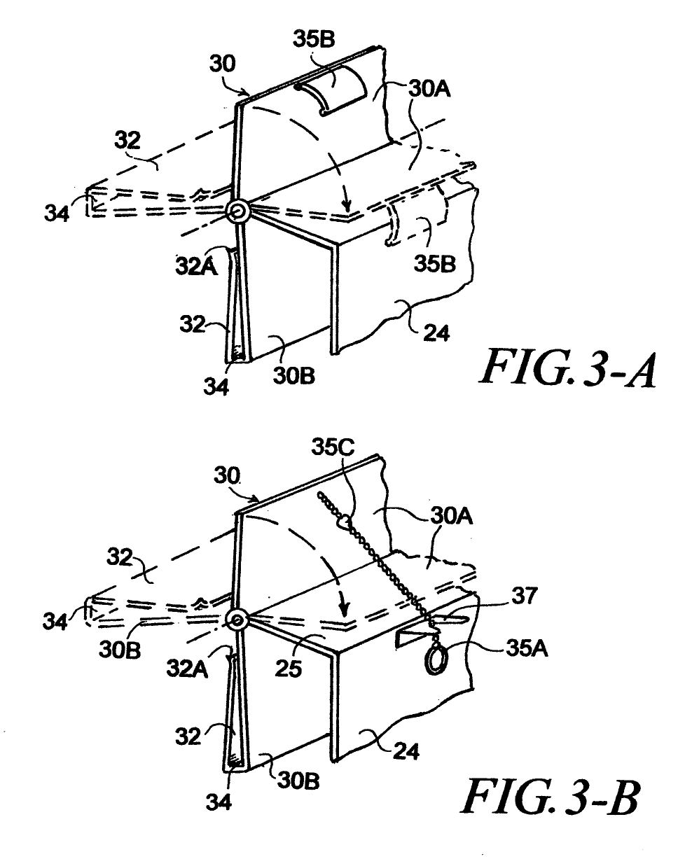

FIGS. 3-A,B is perspective view of two different types of means for one hand opening and fixing in charging/discharging positions of the gate/tray;

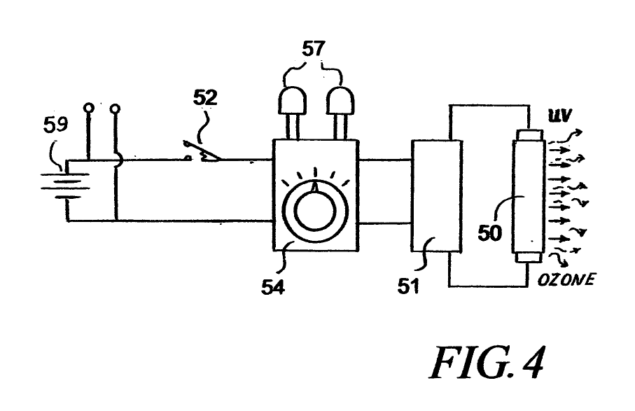

FIG. 4 is a schematic diagram of the electrical supply to UV source and means of information for “start” and “end” of sanitizing procedure;

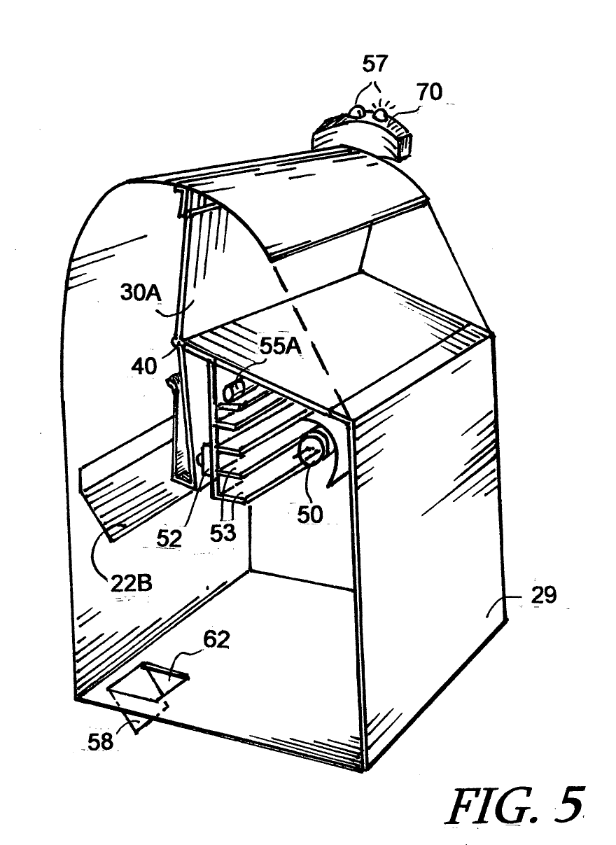

FIG. 5 is a perspective view of the mailbox with fixture for gas-releasing objects and open drain/ventilation aperture;

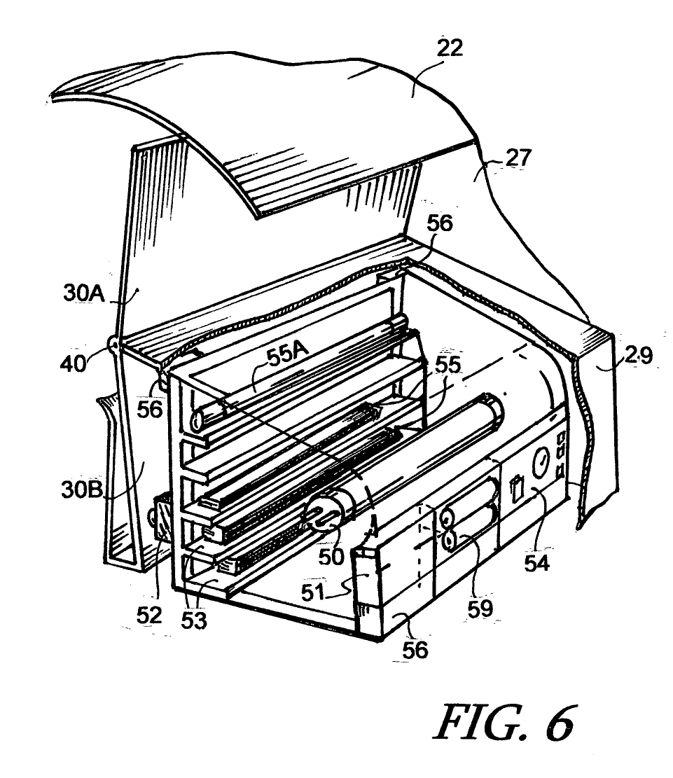

FIG. 6 is a detailed perspective view of the irradiating and gas-generating fixture.

### REFERENCE NUMERALS

20. Sanitizing mailbox

22. Top cylindrical hood of the box

22A. Back wall—extension of the top hood

22B. Cascading lip on the inside of the back wall

23. Positioning frame or stopper on the inlet port

24. Front wall

25. Front hood-wall

26. Left sidewall

27. Right sidewall

28. Bottom of the box

29. Door for retrieving the received mail

29A. Means for locking the door

30. Self closing mail gate/tray with means for fixation of the outgoing mail

30A. Front (visible) part of the gate/tray

30B. Back (hidden) part of the gate/tray

32. Top closing means (page, bracket, etc.) of the outgoing mail pocket

32A. Means for opening the outgoing mail pocket

34. Spine of the outgoing mail pocket

35. Means for forward opening of the gate/tray

35A. Means for fixation of the gate/tray in charging/discharging position

35B. Handle with magnetic means for fixation on the front wall

35C. Member on the rope/chain for fixation the gate in open position

37. Stopper plate for fixing the member 35C in open position

40. Hinge on the main axis of the gate/tray with mail pocket

50. Source of ultraviolet light (UV lamp)

51. Transverter connected to external source of power supply

52. Means for activation of the UV source (button/switch)

53. Holder-beds for sanitizing gas-releasing objects

54. Means for regulating the length of the time activation for the UV source

55. Gas-releasing objects

55 A. Permeation tube

56. Means for holding the UV source, transverter, time relay, reflector and gas-releasing objects in overlooking position—irradiating and gas-generating fixture

57. Means for observation of the duration of activation time and its finish-photodiodes (for example: red—sanitizing activated/green—collect the mail)

58. Drain/ventilation aperture cover

59. Internal source of power supply

60. Means to keep the incoming mail out of contact with the mail box walls; Same means allowing retrieving the mail from the mail box (mail basket)

62. Drain/ventilation aperture

70. Info-flag handle with places for photodiodes

### DETAILED DESCRIPTION OF THE INVENTION

One main embodiment in two different variations is shown and explained on FIGS. 1 to 4. As far as both of the variations have the same aim and are functioning the same way both of the variations are shown in parallel. Parts having the same main function are numbered the same way in all drawings.

As seen on FIGS. 1-A,B and FIGS. 2-A,B the mail box 20 of the present invention is cylindrically hooded secluded box with top port for depositing outgoing and incoming mail and bottom port for retrieving the received mail.

As seen on FIGS. 1-A,B the top port of the mail box 20 is fully closed by the means of top gate/tray 30A and have a retrieving door 29 normally closing the bottom port with means 29A for locking the door to the box. The bottom port closing door is mounted on box front side FIG. 1-A or left/right side FIG. 1-B. It is hanged preferably along its top side, thereby to prevent the box from snow or rain when opened. The box top side is shaped as cylindrical hood 22, which extends partially forward as overhang to prevent the top port and gate/tray 30A from snow or rain. The cylindrical hood 22 extends as vertical back wall 22A of the box 20 adjacent to the bottom wall 28 which is further adjacent to the front wall 24, seen on FIGS. 2-A,B. The top part of the front wall 24 is bent backward forming a sloped wall 25 and preferably has the hinge 40 mounted along its inner edge. The hinge 40 could be replaced by two hinged rivets, as shown on FIGS. 2-A,B. Parallel to the main axis of the cylindrical hood 22 is pivotally hinged the rectangular shaped gate/tray 30 which is closing the top port of the box and which gate/tray is restricted in its rotation to the “closed” position by means of positioning stopper or frame 23, at in its “open’ position by the front sloped wall 25.

The axis of hinge 40 divides the gate/tray 30 in two parts—front or visible part 30A, normally closing the mail box, and its back hidden part 30B inserted into the box 20 and longer inward than front part 30A, seen on FIGS. 2-A,B and FIGS. 3-A,B. Part 30B which is entirely inside of the box is forming a book's hard-cover-like pocket for the outgoing mail by means of the spine 34 adjacent to the back edge of the gate/tray and page 32 adjacent to the other side of the spine 34 and covering the back part 30B of the gate/tray. One or two corners 32A of the page 32 close to the main axis are slightly open up and allow the outgoing mail to be inserted easily into the pocket. The thickness of the pocket and its capacity depends on the width of the spine 34, but should not exceed ¾ of the opening of the incoming port. There is an angle between front part 30A and inserted part 30B of the gate/tray 30 as the surface 30B is bent down along the hinge/axis 40 well seen on FIGS. 2-A,B FIGS. 3-A,B and FIG. 5. The purpose of this bent is to provide a pocket with enough volume and respective capacity for outgoing mail. The other purpose of this bent is to provide planar surface of the gate/tray 30 when page 32 is closed (the outgoing mail is retrieved) and incoming mail have to be charged on the gate/tray 30. When the pocket of the gate/tray is closed the surface of the page 32 have to represent an extension of the plane formed by surface 30A. It is understandable that by enlarging the width of spine 34 the bent of gate/tray must be deeper thereby to keep always flat the entire working surface of the gate/tray for incoming mail, seen on FIGS. 3-A,B.

As shown better on FIGS. 2-A,B when in “closed” position the gate/tray 30 is forming internal hooded space secluded on one side from the back gate/tray 30B, on the top from the sloped front wall 25 and on the third side by the front wall 24. The other two side walls 26 and 27 of the box are defining this hooded space on the top part of which is fixed ultraviolet light source 50 with wave length pick at 254 nanometers or similar. The UV source is mounted in a fixture 56 including other electrical parts and means, shown on FIG. 4, FIG. 5 and FIG. 6, to provide appropriate UV light with power such as: internal source of power supply 59, transverter 51 connected to external power supply, time relay 54, reflector as well as means for mechanical or magnetic fixation to the internal surface. On the bottom 28 of this hooded space is placed basket 60 for the incoming mail. The basket has its grid-type walls sloped inward touching the internal box surface only with the top edges of its walls. A legs situated preferably in the corners of the mail basket do not allow the incoming mail when dropped to touch the bottom of box 20. The distance defined by the legs of the basket is preferably over ¾ inch. The bottom 28 is slightly sloped to one of the sidewalls having in its lowest part means—vent/draining aperture 62 normally closed by cover 58. This aperture allows ventilating by diffusion inner space before opening the door and draining eventually collected water. The basket 60 provides a full air gap wrapping the incoming mail suspended in the mentioned hooded space. A holder with shelves 53 for gas-releasing objects 55 capable of generating sanitizing gas (such as chlorine dioxide, ethylene oxide or similar), when irradiated by light, is placed close to the UV source whereby to increase the disinfecting power of the box. Sodium hypochlorite and sodium chlorate in combination are used to generate “in situ” chlorine dioxide. Permeation tube with ethylene dioxide is used to generate ethylene dioxide.

### Function of the Preferred Embodiment

The source of short wave ultraviolet light is known to generate ozone in the air, in this case into the limited air volume of the mailbox. When acting the UV source is providing UV radiation having direct bactericide (germicidal) effect. The UV radiation is generating also proportional to the source power and time of its work concentration of ozone, which diffuses freely around the inserted mail. The present invention alleviates free diffusion of the ozone around the mail allowing it to be exposed to direct UV light shown in one preferred embodiment on FIGS. 2-A,B. As the basket 60 has its side grids sloped to the inner side of the box walls, they provide needed distance for ozone to diffuse around, as well as the corner legs which provide space between the mail charged in the basket and the bottom 28 of the box. This way the mail is suspended and not contacting eventually condensed or cascaded water on the inner surface of the box and is fully wrapped in air with sufficient concentration of ozone when UV source 50 is activated. In closed position the inner volume of mailbox with volume of about 30 liters the 8 wt UV lamp with 253 nm wavelength (type G8T5) generates up to 8 ppm ozone after half hour exposure and 14 ppm after 1.5 hrs (measured with passive monitors and results extrapolated).

Another preferred embodiment comprises holder-beds 53 containing gas-releasing objects 55 and permeation tube 55A seen on FIG. 5 and in more details on FIG. 6. These objects are activated for the time when UV source irradiates the inner space of the box and release or generate another type sanitizing gases—chlorine, chlorine dioxide and ethylene oxide. The mixture of sanitizing gases in comparably low concentration (1 to 10 ppm) is capable to kill all microorganisms inside the box. A saturated solution of alkaline hypochlorite impregnated into body of porous material when irradiated releases chlorine and hypochloric mist. Both of them react with alkaline chlorate (or mixture of chlorate and perchlorate) incrusted into separate body of porous material, to chlorine dioxide. Each molecule of chlorine generates two molecules of chlorine dioxide. This is the reason to define gas-releasing objects as interacting means in the gas-generating fixture. Only part of chlorine reacts to chlorine dioxide, the other part remains unchanged capable directly to chlorinate or indirectly to oxidize, whereby to destroy biological objects of any kind. The final result is a very powerful reacting and oxidizing germicidal gas mixture generated by the irradiating fixture. To add ethylene oxide to the mixture, a permeation tube 55A (Teflon tube with high pressurized ethylene oxide) is used. The ethylene oxide emission is proportional to the temperature and it is sufficient at elevated temperature when irradiated by the UV source. The gas mixture can be easily diluted for safety by opening aperture 62. The box is completely ventilated by incoming air when door 29 and cover 58, both are open within several seconds. The gas-releasing objects 55 and 55A are made preferably from macro porous material (such as foamed polyurethane, polyethylene, silicone, polysilicates, macro porous stone, ceramic, sintered materials, perlite or alike). One object is impregnated with alkaline hypochlorite; another is incrusted with alkaline chlorate and perchlorates to over 30% of their volume. The volume of each impregnated or incrusted object for 30 L free volume of the mailbox is approximately 200 cc.

In both of the aforementioned preferred embodiments there are means to activate the UV source when the gate/tray 30 is in “closed” position. Button/switch 52 is mounted on the fixture holding the UV source 50 or on the surface of the sloped wall 25. The button/switch 52 is pressed when gate/tray is completely closed by the gravity because the weight of gate/tray part 30B plus the weight of the spine 34 and page 32, is much bigger than the weight of the visible front part 30A, therefore the pocket on the gate/tray 30 is inclined to rotate the gate always into “closed” position, seen on FIGS. 2-A,B. Once activated the UV source 50 generates directly UV light, heat and ozone and indirectly chlorine, chlorine dioxide and ethylene oxide for the predetermined time (preferably over 0.5 hour). The time is regulated by the means of timing relay assembly 54 as shown on FIG. 4 and FIG. 6. The time of activation is indicated by LED indicators 57 mounted on the information flag 70 (preferably red colored for active UV and green colored when the mail can be collected). The electric supply for the UV source could be based on build in or rechargeable battery 59, or permanent DC supplies 51 by external source. For safety reason DC supply preferably to 6-12 volts could be separated from the mailbox.

On FIGS. 1-A,B two variants of one embodiment are shown. The variant on FIG. 1-B is designed for standard envelopes and wraps to be introduced lengthwise on the gate/tray. The variant on FIG. 1-A is more convenient for bigger envelopes and packages to be introduced widthwise on the gate/tray. In both cases the gate/tray serve as self-closing door securing at the same time the dropped mail from theft.

In all variants of the present invention the means for “one hand” service for incoming and outgoing mail are used as shown on FIGS. 3-A,B. Those means provide temporary fixation of the gate/tray in “open” position. They can be based on magnetic fixed lock FIG. 3-A or chain with means for temporary fixation FIG. 3-B.

Materials

No open metal surfaces have to be used all over the inside of the box 20. The ozone has tendency to recombine its atoms on metal surfaces forming three molecules of oxygen from every two molecules of ozone. The presence of open metal surface can deplete completely ozone concentration. On the other hand ozone is strong oxidizer and can contribute to the corrosion on open metal surfaces. Chlorine and chlorine dioxide are strong oxidizers and combined with ozone can easily corrode most of the metals and some of widely used plastics.

All materials used inside the box of the present invention must be corrosion resistant to UV, ozone, chlorine and chlorine dioxide. Well suited are plastic coated metal surfaces as well as plastics recommended for outdoor use—modified PVC, modified acrylic, polyurethane, polycarbonates etc. The best materials are poly fluorinated plastics or materials laminated with poly fluorinated film (Teflon-coated, PTFE-coated, etc).

As UV source any germicidal lamp dimensionally suitable could be used, but lamps with low consumption designed for portable units and use such as types G4T5, G6T5, G8T5 are preferable. A standard or market available electric transverter, usually coming as a part of the portable unit's body, is the power source directly supplying the UV lamp. For convenience the time relay, determining the activation time for the UV source and the transverter, are preferably combined in one body.

Safety

The ozone is a gas with specific odor—three atoms molecule modification of the oxygen that is not very stable and tends to form oxygen allowing one atom of free oxygen, which is extremely active either to recombine with another atom oxygen-to-oxygen gas molecule or to oxidize rapidly many other materials including biological objects. Those properties are the base of the use of ozone as powerful germicidal gas destroying bacteria, spores and viruses of different origin. The ozone is a natural part of the ambient ground-close atmosphere and its concentration varies in large scale (compare to other atmosphere gases), range from 10 to 60 ppb. A small concentration of ozone is considered healthful as a natural sanitizing agent.

The ozone concentration achievable in the safety box is about 5-10 ppm after 0.5 hr activation of the UV source. Even if the mail box is opened immediately after such level is achieved, the concentration will drop instantly several hundred times for a few seconds, because of the big diffusion coefficient of the ozone, big door opening and high volume of surrounding air—thousands times overwhelming the box volume and providing extremely high gradient of concentration depletion. The amount of ozone generated and immediately diluted to the normal atmosphere concentration is negligible even assuming simultaneous work of millions sources alike. Therefore the sanitizing mailbox can be considered as environmental complying device.

In case when other than ozone gases are generated such as chlorine, chlorine dioxide and ethylene oxide, the mailbox should be ventilated by opening the cover 58 on the aperture 62 for several minutes before opening the front door 29 and retrieving the mail. Mailbox using gas-mixture must be placed in open well-ventilated area.

It should be understood that it is in the spirit of the present invention to provide a mail box with sanitizing means by generating short wave UV radiation, ozone and other reactive gases simultaneously and to wrap in this atmosphere any mail for the time considerable enough for the gas to penetrate into and to extinct all microorganisms and spores. At the same time the box upon the present invention is providing secured space for the incoming mail and means for one hand operation to drop the incoming mail and/or pick up the outgoing mail. The box is secured from theft and influence by the elements—rain, snow, cascading water, ice etc.

What we claim is:

1. A secure mail box comprising: a top port and a bottom port; a horizontal pivotal gate with limited angle of rotation, said gate including a substantially flat front part and a back part shaped to define a pocket for holding outgoing mail; said top port normally closed by said front part of said gate and said bottom port normally closed by a door, said door including a locking means; a basket for receiving incoming mail dropped therein from said gate, said basket shaped to provide compete air gap around the received mail; a source of short wave UV light mounted in a fixture, said fixture including a means for reflecting said UV light; a means for electrically powering said source of UV light for a predetermined time; a means for activating said source of UV light when said top port is closed by said gate whereby to expose the mail within the mail box to UV radiation and simultaneously generated ozone; a means to generate chlorine, hypochlorites mist and chlorine dioxide upon irradiation with said UV source; a means to generate ethylene oxide; and a flag having two light indicators on a top surface thereof for indicating activation of said source of UV light and non-activation of UV light.

2. The mailbox of claim 1, where said means to generate chlorine and hypochlorites mist comprises porous inert material impregnated with concentrated solution of alkaline hypochlorite.

3. The mailbox of claim 1 and claim 2, where said means to generate chlorine dioxide comprises inert porous material incrusted with alkaline chlorate.

4. The mailbox of claim 1 and claim 2 where said means to generate chlorine dioxide comprises inert porous material incrusted with mixture of alkaline chlorate and alkaline perchlorate.

5. The mailbox of claim 1, where said means to generate ethylene oxide comprises permeation tube of poly fluorinated plastic filed with high pressurized ethylene oxide.

6. The mailbox of claim 1, where said irradiating fixture is secured in place by strong permanent magnets on the plastic laminated steel inner surface of said box.

7. The mailbox of claims 1,2,3,4,5 and 6 wherein all inside surfaces of said mailbox are made from or are covered by ozone, chlorine and chlorine dioxide compatible materials whereby said surfaces will not interfere with said generated ozone to cause recombination to oxygen and with said generated chlorine and chlorine dioxide to cause corrosion.

8. The mailbox of claim 1,2,3,4,5,6 and 7 wherein said gate is extended to said back part shaped to define pocket and bended backward forming planar surface for deposited incoming mail.
